(19) United States
(12) Patent Application Publication
(10) Pub. No.: US 2002/0035105 A1
(43) Pub. Date: Mar. 21, 2002

(54) Composition and method combining an antidepressant with an NMDA receptor antagonist, for treating neuropathic pain

(76) Inventor: Frank S. Caruso, Colts Neck, NJ (US)

Correspondence Address: Peter G. Dilworth, Esq., DILWORTH & BARRESE, LLP, 333 Earle Ovington Blvd., Uniondale, NY, 11553, US

(21) Appl. No.: 09/966,975

(22) Filed: Sep. 28, 2001

Related U.S. Application Data
(62) Division of application No. 09/434,907, filed on Nov. 5, 1999.
Application No. 09/434,907 is a continuation of application No. PCT/US98/09253, filed on May 6, 1998.
(60) Provisional application No. 60/045,900, filed on May 7, 1997.

(51) Int. Cl. A61K 31/5513; A61K 31/496; A61K 31/135

(52) U.S. Cl. 514/220; 514/253.04; 514/649; 514/657

(57) ABSTRACT

The neuropathic pain alleviating effectiveness of an antidepressant is significantly potentiated by administering the antidepressant prior to, with or following the administration of a nontoxic NMDA receptor antagonist.

### BACKGROUND OF THE INVENTION

This invention relates to a composition and method for alleviating neuropathic pain. More particularly, this invention is directed to such a composition and method in which an antidepressant is combined with a nontoxic antagonist, or blocker, for the N-methyl-D-aspartate (NMDA) receptor.

Neuropathic pain is pain that is due to functional abnormalities of the nervous system. Fields, “Pain”, McGraw-Hill, Inc. (1987), pp. 133 et seq. There are a variety of possible mechanisms by which nerve dysfunction can cause neuropathic pain: hyperactivity in primary afferent or central nervous system (CNS) nociceptive neurons, loss of central inhibitory connections, and increased activity in sympathetic efferents. Neuropathic pain typically occurs following injury to elements of the nervous system involved in nociception, such as peripheral nerve injury, in which the lesions deafferent the nociceptive pathway, the resultant pain sometimes being referred to deafferentation pain. Neuropathic pain is much more likely to occur with peripheral than with central nervous system damage. Examples of causes of painful nerve injury are: accidental trauma, tumors, cerval or lumbar spine disease, and surgical procedures. These injuries usually involve one or two peripheral nerves or nerve roots, and the pain is felt in the body region normally innervated by the damaged nerves. Additionally, there are also toxic, metabolic, and hereditary causes of painful polyneuropathies, erg., alcohol abuse, diabetes mellitus and toxicity due to cancer chemotherapy. These tend to be symmetrical and are most severe on the distal limbs.

U.S. Pat. No. 5,352,683 discloses a method for the treatment of chronic pain, inclusive of neuropathic pain, by administration of a nontoxic N-methyl-D-aspartate receptor antagonist such as dextromethorphan. However, there is no mention in this patent of combining a nontoxic NMDA receptor antagonist with an antidepressant for the treatment of neuropathic pain.

### SUMMARY OF THE INVENTION

In accordance with the present invention, a therapeutic composition for alleviating neuropathic pain is provided which comprises at least one antidepressant in an amount sufficient to alleviate neuropathic pain and at least one nontoxic N-methyl-D-aspartate receptor antagonist in an amount sufficient to potentiate the neuropathic pain-alleviating activity of the antidepressant.

Further in accordance with the present invention, a method of alleviating neuropathic pain is provided which comprises coadministering to a mammal exhibiting neuropathic pain at least one antidepressant in an amount sufficient to alleviate neuropathic pain and at least one nontoxic N-methyl-D-aspartate receptor antagonist in an amount sufficient to potentiate the neuropathic pain-alleviating activity of the antidepressant.

The term “N-methyl-D-aspartate receptor” shall be understood to include all of the binding site subcategories associated with the NMDA receptor, e.g., the glycine-binding site, the phenylcyclidine (PCP)-binding site, etc., as well as the NMDA channel. Thus, the invention herein contemplates the use of nontoxic substances that block an NMDA receptor binding site, e.g., dextromethorphan, or block the NMDA channel, e.g., a source of magnesium such as magnesium sulfate.

The term “nontoxic” as used herein shall be understood in a relative sense and is intended to designate any substance that has been approved by the United States Food and Drug Administration (“FDA”) for administration to humans or, in keeping with established regulatory criteria and practice, is susceptible to approval by the FDA for administration to humans. The term “nontoxic” is also used herein to distinguish the NMDA receptor antagonists, or blockers, that are useful in the practice of the present invention from NMDA receptor antagonists such as MK 801 (the compound 5-methyl-10,11-dihydro-SH-dibenze[a,d] cyclohepten-5,10-imine), CPP (the compound 3-[2-carboxypiperazin4-yl] propyl-1-phosphonic acid) and PCP (the compound 1-(1-phenylcyclohexyl)piperidine) whose toxicities effectively preclude their therapeutic use.

The terms “potentiate” and “potentiating” are used herein in their art-recognized sense, i.e., as referring to a significant increase in the level of neuropathic pain-alleviating activity for the combination of antidepressant and nontoxic NMDA receptor antagonist compared with that which could have been expected based on the neuropathic pain-alleviating activities of the antidepressant and nontoxic NMDA receptor antagonist administered alone.

The expression “neuropathic pain-alleviating” shall be understood herein to include the expressions “neuropathic pain-suppressing” and “neuropathic pain-inhibiting” as the invention is applicable to the alleviation of existing neuropathic pain as well as the suppression or inhibition of neuropathic pain which would otherwise ensue from an imminent neuropathic pain-causing event.

The expression “neuropathic pain-alleviating amount” as applied to the antidepressant employed in the therapeutic composition and method of this invention shall be understood to mean an amount of antidepressant which when administered by itself or in combination with the nontoxic NMDA receptor antagonist provides significant neuropathic pain-alleviating activity.

### DESCRIPTION OF THE PREFERRED EMBODIMENTS

Any of the known and conventional neuropathic pain-alleviating antidepressants can be used herein. For extensive listings of antidepressants, see, e.g., Goodman and Gilman's “The Pharmaceutical Basis Of Therapeutics”, 8th ed., McGraw-Hill, Inc. (1990), pp. 405-414, and “Remington's Pharmaceutical Sciences”, 17th ed., Mack Publishing Company (1985), pp. 1093-1098. Specific neuropathic pain-alleviating antidepressants that can be used herein include tricyclic antidepressants such as imipramine hydrochloride, imipramine pamoate, 2-chloroimipramiine, amitriptyline hydrochloride, amoxapine, desipramine hydrochloride, doxepin hydrochloride, protriptyline hydrochloride, trimipramine maleate, nortriptyline hydrochloride, clomipramine hydrochloride, and the like; tetracyclic antidepressants such as maprotiline hydrochloride, and the like; monoamine oxidase (MAO) inhibitors such as phenelzine sulfate, isocarboxazid, tranylcypromine sulfate, and the like; serotonin uptake inhibitors such as paroxetine hydrochloride, fluoxetine hydrochloride, trazodone hydrochloride, citalopram, cis-isomeric derivative of 4-phenyl-1,2,3,4-tetrahydro-1-naphthalenamine such as sertraline hydrochloride, trans-isomeric derivative of 4-phenyl-1,2,3,4-tetrahydro-1-naphthalenamine, aryloxy indanarnine, and the like; and, other antidepressants such as bupropion hydrochloride and benactyzine hydrochloride.

Among the nontoxic substances that block the NMDA receptor and as such are useful for potentiating the neuropathic pain-alieviating activity of the antidepressant in accordance with this invention are dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), their mixtures and their pharmaceutically acceptable salts. Other useful nontoxic substances that block the NMDA receptor include pyrroloquinoline quinone, 4-hydroxy-2(1H)-quinolone derivatives and cis4-(phosphono-methyl)-2-piperidinecarboxylic acid. Of the foregoing nontoxic substances that block the NMDA receptor, dextromethorphan is preferred due to its ready availablilty and its established use in over-the-counter medications where it functions as a cough suppressant.

The antidepressant must be present in a neuropathic pain-alleviating amount. Such an amount can correspond to the recommended adult human dosage level for a particular antidepressant when administered by itself or it can be less than this amount provided that in combination with the nontoxic NMDA receptor antagonist, significant neuropathic pain-alleviating activity is achieved. The NMDA receptor antagonist must be present at a level sufficient to potentiate the neuropathic pain-alleviating effectiveness of the antidepressant. Specific dosage levels for the antidepressants that can be used herein are given, inter alia, in the “Physicians' Desk Reference”, 1996 Edition (Medical Economics Data Production Company, Montvale, N.J.) as well as in other reference works including Goodman and Gilman's “The Pharmaceutical Basis of Therapeutics” and “Remington's Pharmaceutical Sciences” both of which as referred to above. Given the wide variation in dosage level of the antidepressant which depends to a large extent on the specific antidepressant being administered, there can similarly be a wide variation in the dosage level of the NMDA receptor antagonist. These amounts can be determined for a particular drug combination employing routine experimental testing. For example, in the case of the tricyclic antidepressant imipramine hydrochloride and the nontoxic NMDA receptor blocker dextromethorphan, dosages of from about 50 to about 360 mg/day of the former coadministered with from about 30 to about 120 mg/day of the latter will usually provide acceptable results.

When the nontoxic NMDA receptor antagonist selected for use herein is dextromethorphan, dextrorphan or sale thereof, the antidepressant drug must be other than a monoamine oxidase inhibitor since antidepressants of this type are contraindicated for these NMDA receptor antagonists.

While the neuropathic pain-alleviating antidepressant and the potentiating nontoxic NMDA receptor antagonist need not be administered together, they must both be present in the patient at effective levels at the same time. While it is within the scope of the invention to separately administer the antidepressant and the nontoxic NMDA receptor antagonist, as a matter of convenience, it is preferred that these drugs be coadministered in a single dosage form. All modes of administrations are contemplated, e.g., orally, rectally, parenterally, nasally, topically or by intravenous or intramuscular injection.

A therapeutic composition containing the antidepressant and nontoxic NMDA receptor antagonist will ordinarily be formulated with one or more pharmaceutically acceptable ingredients in accordance with known and established practice. Thus, the composition can be formulated as a liquid, powder, elixir, injectable solution, etc. Formulations for oral use can be provided as tablets or hard capsules wherein the pharmacologically active ingredients are mixed with an inert solid diluent such as calcium carbonate, calcium phosphate or kaolin, or as soft gelatin capsules wherein the active ingredients are mixed with an oleaginous medium, e.g., liquid paraffin or olive oil.

Aqueous suspensions can include pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkylene oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. The aqueous suspensions can also contain one or more preservatives, e.g., ethyl-or-n-propyl-p-hydroxy benzoate, one or more coloring agents, one or more flavoring agents and one or more sweetening agents, such as sucrose, saccharin or sodium or calcium cyclamate.

In addition to the antidepressant and nontoxic NMDA receptor antagonist, the therapeutic composition herein can optionally contain at least one other pharmacologically active substance e.g., an antianxiety agent such as meprobamate and benzodiazepines, e.g., chlordiazepoxide, diazepam, oxazepam, clorazepate, lorazepam, prazepam, alprazolam, halazepam, clonazepam and the like; an antipsychotic agent such as phenothiazines, e.g., perphenazine, chlorpromazine hydrochloride, triflupromazine hydrochloride, mesoridazine besylate, thioridazine hydrochloride, acetophenazine maleate, fluphenazine hydrochloride, fluphenazine enanthate, fluphenazine decanoate, trifluoperazine hydrochloride and the like; a non-narcotic analgesic such as tramadol, acetaminophen, aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac and the like; or a narcotic analgesic such as codeine, dihydrocodeine, hydrocodone, levorphanol, morphine, oxycodone, and the like.

### EXAMPLES 1-46

The following unit dosage forms are illustrative of the neuropathic pain-alleviating therapeutic combinations in accordance with the present invention:

| Ex- | | | Nontoxic | Active Additional |
|---|---|---|---|---|
| am- | Dosage | Antidepressant | NMDA Receptor | Component(s) |
| ple | Form | Drug (mg) | Blocker (mg) | (mg) |
| 1 | capsule | chlomipramine | dextromethorphan | |
| | | hydrochloride | hydrobromide | |
| | | (25) | (30) | |
| 2 | capsule | chlomipramine | dextromethorphan | |
| | | hydrochloride | hydrobromide | |
| | | (50) | (30) | |
| 3 | capsule | imipramine | dextromethorphan | |
| | | pamoate (75) | hydrobromide | |
| | | | (30) | |
| 4 | capsule | imipramine | dextromethorphan | |
| | | pamoate (125) | hydrobromide | |
| | | | (30) | |
| 5 | tablet | imipramine | dextromethorphan | |
| | | hydrochloride | hydrobromide | |
| | | (50) | (30) | |
| 6 | intra- | imipramine | dextromethorphan | |
| | muscular | hydrochloride | hydrobromide | |
| | injection | (100) | (30) | |
| 7 | tablet | amoxapine (50) | dextromethorphan | |
| | | | hydrobromide | |
| | | | (30) | |
| 8 | tablet | desipramine | dextromethorphan | |
| | | hydrochloride | hydrobromide | |
| | | (100) | (30) | |
| 9 | tablet | amitriptyline | dextromethorphan | perphenazine |
| | | hydrochloride | hydrobromide | (2) |
| | | (25) | (30) | |
| 10 | tablet | amitriptyline | dextromethorphan | chlordiazepoxide |
| | | hydrochloride | hydrobromide | (10) |
| | | (25) | (30) | |
| 11 | capsule | doxepin | dextromethorphan | |
| | | hydrochloride | hydrobromide | |
| | | (25) | (30) | |
| 12 | capsule | nortriptyline | dextromethorphan | |
| | | hydrochloride | hydrobromide | |
| | | (25) | (30) | |
| 13 | tablet | amitriptyline | dextromethorphan | |
| | | hydrochloride | hydrobromide | |
| | | (75) | (30) | |
| 14 | intra- | amitriptyline | dextromethorphan | |
| | muscular | hydrochloride | hydrobromide | |
| | injection | (20) | (30) | |
| 15 | capsule | trimipramine | dextromethorphan | |
| | | maleate (25) | hydrobromide | |
| | | | (30) | |
| 16 | tablet | fluoxetine | dextromethorphan | |
| | | hydrochloride | hydrobromide | |
| | | (10) | (30) | |
| 17 | tablet | sertraline | dextromethorphan | |
| | | hydrochloride | hydrobromide | |
| | | (50) | (30) | |
| 18 | tablet | paroxetine | dextromethorphan | |
| | | hydrochloride | hydrobromide | |
| | | (20) | (30) | |
| 19 | tablet | maprotiline | dextromethorphan | |
| | | hydrochloride | hydrobromide | |
| | | (50) | (30) | |
| 20 | tablet | trazodone | dextromethorphan | |
| | | hydrochloride | hydrobromide | |
| | | (50) | (30) | |
| 21 | tablet | bupropion | dextromethorphan | |
| | | hydrochloride | hydrobromide | |
| | | (100) | (30) | |
| 22 | tablet | benactyzine | dextromethorphan | meprobamate |
| | | hydrochloride | hydrobromide | (400) |
| | | (1) | (30) | |
| 23 | tablet | imipramine | dextrorphan | |
| | | hydrochloride | hydrobromide | |
| | | (25) | (30) | |
| 24 | tablet | fluoxetine | dextrorphan | |
| | | hydrochloride | hydrobromide | |
| | | (10) | (30) | |
| 25 | tablet | imipramine | amantadine (30) | |
| | | hydrochloride | | |
| | | (25) | | |
| 26 | tablet | fluoxetine | amantadine (30) | |
| | | hydrochloride | | |
| | | (20) | | |
| 27 | tablet | imipramine | memantine (30) | |
| | | hydrochloride | | |
| | | (25) | | |
| 28 | tablet | fluoxetine | memantine (30) | |
| | | hydrochloride | | |
| | | (25) | | |
| 29 | tablet | imipramine | dextromethorphan | acetaminophen |
| | | hydrochloride | hydrobromide | (325) |
| | | (25) | (30) | |
| 30 | tablet | imipramine | dextromethorphan | aspirin (325) |
| | | hydrochloride | hydrobromide | |
| | | (25) | (30) | |
| 31 | tablet | imipramine | dextromethorphan | ibuprofen (325) |
| | | hydrochloride | hydrobromide | |
| | | (25) | (30) | |
| 32 | tablet | fluoxetine | dextromethorphan | acetaminophen |
| | | hydrochloride | hydrobromide | (325) |
| | | (10) | (30) | |
| 33 | tablet | fluoxetine | dextromethorphan | aspirin (325) |
| | | hydrochloride | hydrobromide | |
| | | (10) | (30) | |
| 34 | tablet | fluoxetine | dextromethorphan | ibuprofen (325) |
| | | hydrochloride | hydrobromide | |
| | | (10) | (30) | |
| 35 | tablet | imipramine | dextrorphan | acetaminophen |
| | | hydrochloride | hydrobromide | (325) |
| | | (25) | (30) | |
| 36 | tablet | imipramine | dextrorphan | aspirin (325) |
| | | hydrochloride | hydrobromide | |
| | | (25) | (30) | |
| 37 | tablet | imipramine | amantadine (30) | acetaminophen |
| | | hydrochloride | | (325) |
| | | (25) | | |
| 38 | tablet | fluoxetine | amantadine (30) | aspirin (325) |
| | | hydrochloride | | |
| | | (70) | | |
| 39 | tablet | imipramine | memantine (30) | acetaminophen |
| | | hydrochloride | | (325) |
| | | (25) | | |
| 40 | tablet | imipramine | memantine (30) | ibuprofen (325) |
| | | hydrochloride | | |
| | | (25) | | |
| 41 | tablet | phenelzine | amantadine (30) | |
| | | sulfate (15) | | |
| 42 | tablet | phenelzine | memantine (30) | |
| | | sulfate (15) | | |
| 43 | tablet | isocarboxazid | amantadine (30) | |
| | | (30) | | |
| 44 | tablet | isocarboxazid | memantine (30) | |
| | | (30) | | |
| 45 | tablet | tranylcypro- | amantadine (30) | |
| | | mine sulfate | | |
| | | (30) | | |
| 46 | tablet | tranylcypro- | memantine (30) | |
| | | mine sulfate | | |
| | | (30) | | |

In each of these dosage units, the nontoxic NMDA receptor antagonist dextromethorphan hydrobromide significantly potentiates the neuropathic pain-alleviating activity of the antidepressant component(s).

What is claimed is:

1. A therapeutic composition comprising at least one antidepressant, in an amount sufficient to alleviate neuropathic pain and at least one nontoxic N-methyl-D-aspartate receptor antagonist in an amount sufficient to potentiate the neuropathic pain-alleviating activity of the antidepressant.

2. The therapeutic composition of claim 1 wherein the antidepressant is at least one member selected from the group consisting of tricyclic antidepressants, tetracyclic antidepressants, monoamine oxidase inhibitors, serotonin uptake inhibitors, bupropion hydrochloride and benactyzine hydrochloride.

3. The therapeutic composition of claim 2 wherein the tricyclic antidepressant is selected from the group consisting of imipramine hydrochloride, uinipramine pamoate, 2-chloroimipramine, amitriptyline hydrochloride, amoxapine, desipramine hydrochloride, doxepin hydrochloride, protriptyline hydrochloride, trimipramine maleate, nortriptyline hydrochloride and clomipramine hydrochloride.

4. The therapeutic composition of claim 2 wherein the tetracyclic antidepressant is maprotiline hydrochloride.

5. The therapeutic composition of claim 2 wherein the monoamine oxidase inhibitor is selected from the group consisting of phenelzine sulfate, isocarboxazid and tranylcypromine sulfate.

6. The therapeutic composition of claim 2 wherein the serotonin uptake inhibitor is selected from the group consisting of paroxetine hydrochloride, fluoxetine hydrochloride, trazodone hydrochloride, citalopram, cis-isomeric derivative of 4-phenyl-1,2,3,4-tetrahydro-1-naphthalenamine, trans-isomeric derivative of 4-phenyl-1,2,3,4-tetrahydro-1-naphthalenamine and aryloxy indananline.

7. The therapeutic composition of claim 1 containing a therapeutically effective amount of at least one other pharmacologically active substance.

8. The therapeutic composition of claim 7 wherein the other pharmacologically active substance is selected from the group consisting of antianxiety agents, antipsychotic agents, non-narcotic analgesics and narcotic analgesics.

9. The therapeutic composition of claim 8 wherein the antianxiety agent is selected from the group consisting of meprobamate and chlordiazepoxide.

10. The therapeutic composition of claim 8 wherein the antipsychotic agent is perphenazine.

11. The therapeutic composition of claim 8 wherein the non-narcotic analgesic is selected from the group consisting of tramadol, acetaminophen, aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin and zomepirac.

12. The therapeutic composition of claim 8 wherein the narcotic analgesic is selected from the group consisting of codeine, dihydrocodeine, hydrocodone, levorphanol, morphine, and oxycodone.

13. The therapeutic composition of claim 1 wherein the nontoxic NMDA receptor blocker is at least one member selected from the group consisting of dextromethorphan, dextrorphan, amantadine, memantine and pharmaceutically acceptable salt thereof, except that in the case of dextrorphan or salt thereof, the antidepressant is other than of the monoamine oxidase inhibitor type.

14. A method of alleviating neuropathic pain which comprises administering to a mammal exhibiting neuropathic pain at least one antidepressant in an amount sufficient to alleviate neuropathic pain and at least one nontoxic N-methyl-D-aspartate receptor antagonist in an amount sufficient to potentiate the neuropathic pain-alleviating activity of the antidepressant.

15. The method of claim 14 wherein the antidepressant is at least one member selected from the group consisting of tricyclic antidepressants, tetracyclic antidepressants, monoamine oxidase inhibitors, serotonin uptake inhibitors, bupropion hydrochloride and benactyzine hydrochloride.

16. The method of claim 15 wherein the tricyclic antidepressant is selected from the group consisting of imipramine hydrochloride, imipramine pamoate, 2-chloroimipramine, amitriptyline hydrochloride, amoxapine, desipramine hydrochloride, doxepin hydrochloride, protriptyline hydrochloride, trimipramine maleate, nortriptyline hydrochloride and clomipramine hydrochloride.

17. The method of claim 15 wherein the tetracyclic antidepressant is maprotiline hydrochloride.

18. The method of claim 15 wherein the monoamine oxidase inhibitor is selected from the group consisting of phenelzine sulfate, isocarboxazid and tranylcypromine sulfate.

19. The method of claim 15 wherein the serotonin uptake inhibitor is selected from the group consisting of paroxetine hydrochloride, fluoxetine hydrochloride, trazodone hydrochloride, citalopram, cis-isomeric derivative of 4-phenyl-1,2,3,4-tetrahydro-1-naphthalenamine trans-isomeric derivative of 4-phenyl-1,2,3,4-tetrahydro-1-naphthalenamine, and aryloxy indanamine.

20. The method of claim 14 wherein the nontoxic NMDA receptor blocker is at least one member selected from the group consisting of dextromethorphan, dextrorphan, amantadine, memantine and pharmaceutically acceptable salt thereof, except that in the case of dextromethorphan, dextrorphan or salt thereof, the antidepressant is other than of the monoamine oxidase inhibitor type.
